(19) United States
(12) Patent Application Publication
(10) Pub. No.: US 2011/0214464 A1
(43) Pub. Date: Sep. 8, 2011

(54) BIOCONTROL STRAIN AGAINST THE WILT OF CONTINUOUS CROPPING CUCUMBER AND WATERMELON AND ITS MICROBIAL ORGANIC FERTILIZER

(75) Inventors: Qirong Shen, Nanjing (CN); Lihua Chen, Nanjing (CN); Xingming Yang, Nanjing (CN); Yangchun Xu, Nanjing (CN); Wei Ran, Nanjing (CN); Qiwei Huang, Nanjing (CN); Biao Shen, Nanjing (CN)

(73) Assignee: NANJING AGRICULTURAL UNIVERSITY, Nanjing (CN)

(21) Appl. No.: 12/747,510

(22) PCT Filed: Nov. 23, 2009
(86) PCT No.: PCT/CN09/75076

§ 371 Date: Jun. 10, 2010

(30) Foreign Application Priority Data
Oct. 29, 2009 (CN) 200910233576.1

(51) Int. Cl. C05F 11/08; C12N 1/14

(52) U.S. Cl. 71/8; 435/256.7

(57) ABSTRACT

The present invention relates to a biocontrol strain against the wilt of continuous cropping cucumber and watermelon as well as its microbial organic fertilizer, which belongs to the technology for agricultural intensive production. The present invention separates Trichoderma harzianum SQR-T037, which has a remarkable biocontrol effect against wilt of cucumber and watermelon. Microbial organic fertilizer was produced from this biocontrol strain and organic compost. In this fertilizer, the content of the strain is above 1×10⁸ cfu/g, the content of total nitrogen is 3˜5% (above 90% of the nitrogen is organic nitrogen), total nitrogen-phosphorus-kalium nutrient is 6˜10% and organic matter is 30˜35%. As indicated by the experiments, after this microbial organic fertilizer is applied to soil, it will enable rapid multiplication of biocontrol strain into a dominant microflora in the soil and achieve more than 85% of biocontrol rate of wilt in the soil suffering from serious cucumber and watermelon wilt. If this microbial organic fertilizer is applied to the soil without continuous cropping obstacle for a long time, its controlling rate of cucumber and watermelon wilt will be above 80%.

### FIELD OF THE INVENTION

The present invention relates to a biocontrol strain against the wilt of continuous cropping cucumber and watermelon as well as its microbial organic fertilizer, which belongs to the technology for intensive agricultural production and is dedicated to overcoming and eliminating the wilt of continuous cropping cucumber and watermelon.

### BACKGROUND OF THE INVENTION

Cucumber and watermelon are important economic crops in China, Their yield and quality are related to the national economy and the people's livelihood. Cucumber and watermelon wilt is one of the major diseases of cucumber and watermelon and particularly does serious harm on the planting in protected land. Cucumber wilt was first reported in the Netherlands in 1943. In 1949, it destroyed above 40% of the cucumber in Florida, the USA and infected more than 70% of the continuous cropping plants in the third season. Currently, in the highly intensive cucumber and watermelon growing areas in China, a serious wilt can result in loss of about 90% of yield. Cucumber and watermelon wilt is a soilborne fungal disease. It is widely distributed in the tropical zone, subtropical zone and some warm areas in the world. Fusarium oxysporum—the pathogen of cucumber and watermelon wilt can be spread through soil, flowing water (for example, irrigation), seeds and other means and is highly adaptable to the environment. Once cucumber is infected with Fusarium oxysporum, it will damp off before or after it comes out of the soil, the vine and stem at the top will wither, the vascular bundles at bud base will die and in the end the entire plant will perish. Fusarium oxysporum can result in the damping-off, cortex decay and plant dwarfing of watermelon in seedling stage, and the sudden or gradual wilting of the old plants. Under the condition of high temperature and high humidity, the disease develops rapidly, and the morbidity in the fields with serious attack of wilt is 100%, resulting in no harvest.

On the other hand, the straw of paddy, wheat, corn, rape and other crops is burned on the spot and the excrement of the livestock and poultry raised on a scale is discarded. It not only seriously pollutes environment but also is a great waste of the raw material that can be used to produce organic fertilizer and bio-organic fertilizer products; enormous nutritive resources (C, N, P, K, S and trace elements) are lost outside the soil—plant system and the capability of sustainable development of Chinese agriculture is obviously weakened. How to maximally return the nutritive elements taken away from soil due to harvesting of crops to the soil? The only way is to make these solid organic wastes into commercial organic fertilizer and apply the fertilizer to the soil. If these solid organic wastes are synthesized into high-grade organic compost through high-temperature fermentation and then the compost is used as a carrier of functional bacteria to prepare microbial organic fertilizer, the function will be clear and there will be a good application prospects.

### SUMMARY OF THE INVENTION

### Technical Problem

The purpose of the present invention is to develop an antagonistic microbial organic fertilizer that can prevent and eliminate cucumber and watermelon wilt. The controlling rate of this fertilizer in fields is above 80%. It can biologically restore the soil with continuous cropping obstacle and ensure the smooth development of intensive agriculture.

### Technical Solution

The biocontrol strain SQR-T037 that is used to prevent and eliminate the wilt of continuous cropping cucumber and watermelon belongs to Trichoderma harzianum. It was collected by China General Microbiological Culture Collection Center (CGMCC) on Sep. 22, 2009, and the culture collection number is CGMCC No. 3308. The main biological characteristics are follows: the optimum growth temperature on potato-dextrose-agar (PDA) culture medium is 30° C.; the diameter of colony can be reached 90 mm and it can cover the whole dish and generate yellow pigment in the culture medium when incubated for 96 hours; the conidiophore is in a shape of bottle; the conidium is oval, 2.3-3.5 μm long and 2.0-3.2 μm wide, its surface is smooth. At the beginning color of conidium is yellow green, then it changes into dark green slowly.

In the microbial organic fertilizer produced from the above biocontrol strain that prevents and eliminates the wilt of continuous cropping cucumber and watermelon, the content of SQR-T037 strain is above 1×108 cfu/g, the content of total nitrogen is 4˜5% (weight percent), above 90% of the total nitrogen is organic nitrogen, the content of total nitrogen-phosphorus-kalium nutrient is 6˜10% (weight percent) and the content of organic matter is 30˜35% (weight percent).

The microbial organic fertilizer that prevents and eliminates the wilt of continuous cropping cucumber and watermelon is produced by the following method:
1) SQR-T037 is incubated in PDA culture to conduct liquid fermentation production under the following conditions: the initial pH range for fermentation is 6.5-7.2; the culture temperature is 25-30° C.; DO (dissolved oxygen): ventilation range is 30˜100%, 170 rpm; the fermentation time is 48 h; all mycelium pellets are broken into colony formation units of this strain in the late stage of fermentation, and the colony count of SQR-T037 in fermentation liquor is ≧1×109 cfu/mL; the preparation method of the used PDA culture (taking the preparation of 1 L of culture medium for example): Unpeel 200 g of potato, cut it into small dices, and boil it in boiling water for 30 min, then filter the mixture, add 20 g of common sucrose into the filtrate, fix the volume at 1000 mL, adjust pH value to 6.5-7.0, and sterilize the liquor at 121° C. for 20 min.
2) SQR-T037 fermentation liquor is respectively incubated to the mature livestock or poultry excrement compost and the mixture from microbial enzymatic hydrolysis of rapeseed meal at a dosage of 50 L/t. to conduct solid fermentation. The fermentation temperature is 30-50° C. During the fermentation, the material is turned over once a day. The fermentation is completed in 5-7 days to ensure the colony count of this strain is above 1×10⁸ cfu/g. In the end, the solid microbial agent of biocontrol strain SQR-T037 is obtained; the germination index of the mature livestock or poultry excrement compost is more than 98%; the content of organic matter is ≧35%; the content of organic nitrogen is 1.2-2% and water content is 25-30%.
3) The solid microbial agent SQR-T037 made from livestock or poultry excrement compost (50-80% v/v) and the solid microbial agent SQR-T037 made from the mixture from enzymatic hydrolysis of rapeseed meal (20-50% v/v) are thoroughly mixed. The mixture is ripened 2-3 days. During the ripening, it is turned over twice. In the end, the microbial organic fertilizer is evaporated at temperature of below 50° C. till its water content is less than 30%. After packaging, the microbial organic fertilizer that prevents and eliminates the wilt of continuous cropping cucumber and watermelon is obtained.

The microbial organic fertilizer can be exclusively used to prevent and eliminate the wilt of continuous cropping cucumber and watermelon.

### Beneficial Effects

The present invention relates to a microbial organic fertilizer that can overcome or eliminate the wilt of continuous cropping cucumber and watermelon as well as its producing method. The livestock or poultry excrement compost and the amino acid mixture from enzymatic hydrolysis of rapeseed meal are mixed with the biocontrol strain liquid to produce microbial organic fertilizer. Comparing with the products in the current market, the product has the following advantages:
1) This fertilizer product contains high-performance strain (Trichoderma harzianum) that inhibits the growth of the pathogenic fungi of cucumber and watermelon wilt. The inhibition effect is very remarkable. The experiment result indicates that after this product is applied to the soil suffering from the wilt of continuous cropping cucumber and watermelon, the biocontrol rate of cucumber and watermelon wilt is above 85%, and the biocontrol rate of the soil applied with this fertilizer in three consecutive years is even higher.
2) This fertilizer is bio-organic fertilizer and contains rich organic matter (the content is 30-35%) and organic nitrogen (the content is 3-5%). The content of total phosphor in this product is 2-4%. Moreover, this product has brilliant bioavailability. After this product is used, crops can smoothly get through phosphorin sensitive period in seedling stage. In comparison, after phosphoric fertilizer is applied to soil, the bioavailability is poor. The rich nutrition in bio-organic fertilizer also provides a condition for the growth and multiplication of the biocontrol strain in the fertilizer so that it can survive in soil and form a dominant microflora, thus playing a role in preventing disease.
3) As it is a bio-strain preparation, it doesn't have any problem caused by the use of chemical pesticides and is conducive to the pollution-free production of cucumber and watermelon. Farmers need not use other chemical pesticides that prevent and cure wilt or can reduce the dosage of other chemical pesticides. This not only can reduce farmers' expenditure but also can improve the quality of agricultural products. Meanwhile, this microbial fertilizer has the function of increasing yield, so farmers' revenue can be increased.

### BRIEF DESCRIPTION OF THE DRAWINGS

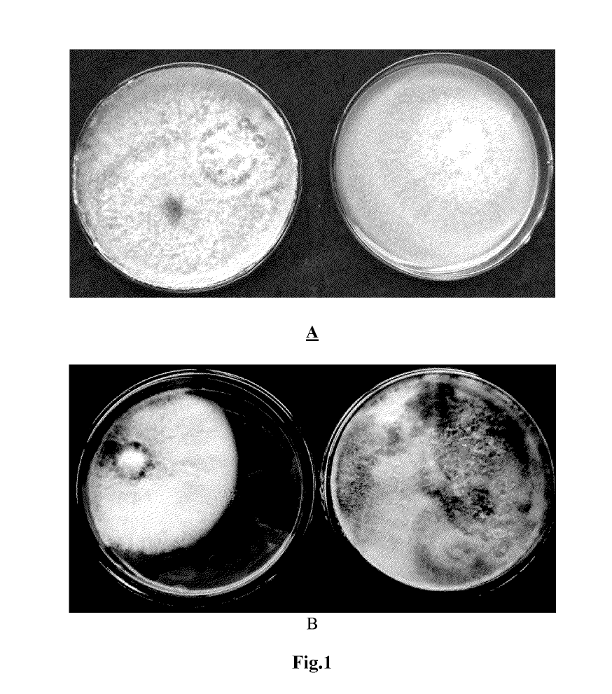

FIG. 1: Effect of biocontrol strain SQR-T037 on inhibiting the pathogen of cucumber and watermelon wilt
A: Effect drawing of the inhibition of SQR-T037 to the pathogen of cucumber wilt
B: Effect drawing of the inhibition of SQR-T037 to the pathogen of watermelon wilt

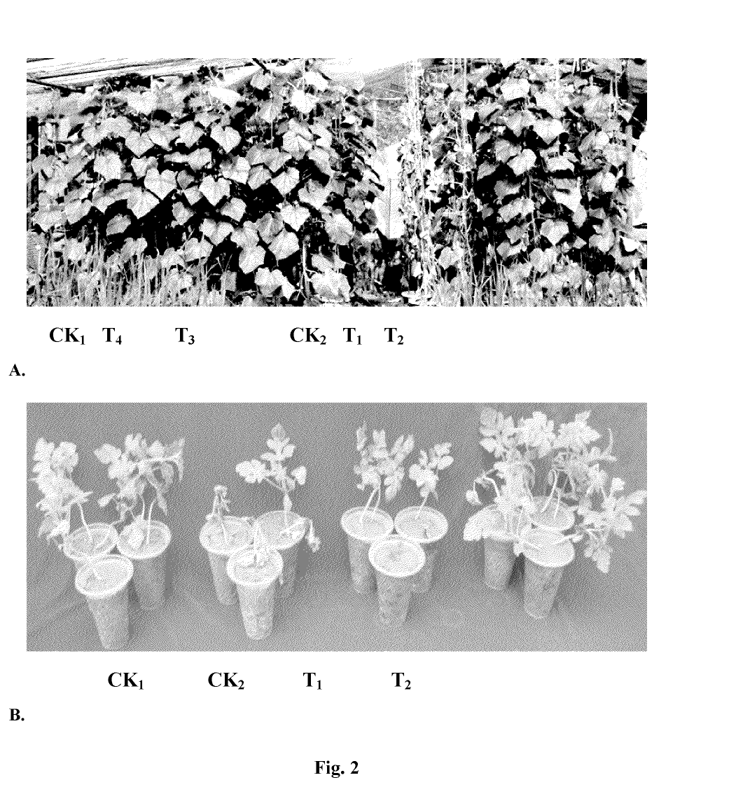

FIG. 2: Controlling effect in greenhouse pot experiment
A: Effect of the bio-organic fertilizer made from SQR-T037 on inhibiting cucumber wilt
CK1: Control; CK2: Soil for continuous cropping of cucumber; T1: CK2+organic fertilizer; T2: CK2+fermentation liquor of Trichoderma SQR-T037; T3: CK2+mixed organic fertilizer of Trichoderma SQR-T037; T4: CK2+organic fertilizer generated from fermentation of Trichoderma SQR-T037
B: Effect of the bio-organic fertilizer made from SQR-T037 on inhibiting watermelon wilt
CK1: Control; CK2: Soil for continuous cropping of watermelon; T1: CK2+organic fertilizer; T2: CK2+Organic fertilizer generated from fermentation of Trichoderma SQR-T037

### DETAILED DESCRIPTION OF THE PREFERRED EMBODIMENT

### Strain Separation and Identification

Samples of the plants with continuous cropping obstacle and serious symptoms of wilt as well as the surrounding soil are collected and stored at low temperature. The selective medium of Fusarium oxysporum is adopted to separate pathogens—Fusarium oxysporum f sp. Cucumerinum and Fusarium oxysporum f sp. niveum. Then the separated pathogenic bacteria of wilt are used as indicators, and the selective medium of Trichoderma is used to separate biocontrol strain from the organic compost that has been composted at high temperature. In the end, through pot experiment and field experiment, high-performance biocontrol strain SQR-T037 is obtained by means of secondary screening. The strain is identified. It is stored in PDA culture media at 4° C. and at room temperature. Biocontrol strain SQR-T037 can fully inhibit the growth of pathogens in 72 h (FIG. 1).

Strain SQR-T037 belongs to Trichoderma harzianum. The main biological characteristics are: the optimum growth temperature on PDA culture medium is 30° C.; the diameter of colony can be reached 90 mm and it will cover the whole dish and generate yellow pigment in the culture medium when incubated for 96 hours; the conidiophore is in a shape of bottle; the conidium is oval, 2.3-3.5 μm long and 2.0-3.2 μm wide, and its surface is smooth; at the beginning its color is yellow green, then it changed into dark green slowly.

### Production of the Bacterial Preparation

1) SQR-T037 is incubated to PDA culture to conduct liquid fermentation production under the following conditions: the initial pH range is 6.5-7.0; the culture temperature is 25° C.; DO: ventilation range is 30˜100% (v/v), 170 rpm; the fermentation time is 48 h; all mycelium pellets are broken into colony formation unit of this strain in the late stage of fermentation, and the colony count (mycelium fragment) of SQR-T037 in fermentation liquor is ≧1×10⁹ cfu/mL;

Preparation method of the used PDA culture (taking the preparation of 1 L of culture medium for example): Unpeel 200 g of potato, cut it into small dices, and boil it in boiling water for 30 min, then filter the mixture, add 20 g of common sucrose into the filtrate, fix the volume at 1000 mL, adjust pH value to 6.5-7.0, and sterilize the liquor at 121° C. for 20 min.

2) SQR-T037 fermentation liquor is incubated to the mature pig excrement compost and the mixture from microbial enzymatic hydrolysis of rapeseed meal at a dosage of 50 L/t. to conduct solid fermentation. During the fermentation, the material is turned over once a day to ensure the fermentation temperature is 30-50° C. The fermentation is completed in 5-7 days to ensure the colony count of the biocontrol strain is above 1×10⁸ cfu/g. In the end, the solid microbial agent of biocontrol strain SQR-T037 is obtained; the germination index of the mature pig excrement compost is more than 98%, the content of organic matter is ≧35%, the content of organic nitrogen is 1.2-2% and water content is 25-30%.

The mixture from microbial enzymatic hydrolysis of rapeseed meal is produced by the following method (known and used by the public, see Chinese invention patent ZL200610086 126.0, a biological preparation method of amino acids for agricultural use and their fertilizer product): add the fermentation liquor of Steno trophomonas maltophilia strain 37-1 into raw material rapeseed meal; adjust water content to 55-65% and pH value to 6.0-7.5; conduct open solid fermentation; turn over it once the fermentation temperature rises to 50° C., turn it over every day since then, and maintain the temperature at 35-50° C. for about 5-7 days. After solid fermentation starts, the pH value of the material will keep rising. Whenever it is turned over, acidic liquid should be added to adjust water content and pH value and maintain water content at 55-65% and pH value at 6.0-7.0. When fermentation is terminated, acidic liquid will be sprayed again till pH value of the material is about 5.0. After that, the material is dried at low temperature or by air. The final product is the mixture from microbial enzymatic hydrolysis of rapeseed meal (mixture containing amino acids).

3) The solid microbial agent SQR-T037 made from pig excrement compost (80% v/v) and the solid microbial agent SQR-T037 made from the mixture from microbial enzymatic hydrolysis of rapeseed meal (20% v/v) are thoroughly mixed. The mixture is ripened 2-3 days. During the ripening, it is turned over twice. In the end, the microbial organic fertilizer is evaporated at temperature of below 50° C. till its water content is less than 30%. Thus the microbial organic fertilizer that controls the wilt of continuous cropping cucumber and watermelon is obtained.

In the microbial organic fertilizer (microbial organic fertilizer that prevents and eliminates the wilt of continuous cropping cucumber and watermelon) generated from the re-fermentation of the above SQR-T037, the colony count of the biocontrol strain SQR-T037 is above 1×10⁸ cfu/g, the content of total nitrogen is 4˜5% (weight percent), above 90% of the total nitrogen is organic nitrogen, total nitrogen-phosphorus-kalium nutrient is 6˜10% (weight percent) and organic matter is 30˜35% (weight percent).

### Greenhouse Pot Experiment

Wilt Prevention and Cure in the Soil for Continuous Cropping of Cucumber

The experimental soil is continuous cropping soil, with serious wilt. The extensively planted “Jin Chun 4” cucumber is adopted. The treatment is as follows:

CK₁: Control; CK₂: Soil for continuous cropping of cucumber; T₁: CK₂+organic fertilizer; T₂: CK₂+fermentation liquor of SQR-T037; T₃: Microbial organic fertilizer produced through simply mixing CK₂+SQR-T037 and organic fertilizer; T₄: Microbial organic fertilizer produced through re-fermentation of CK₂+SQR-T037 and organic fertilizer

Each treatment is repeated five times, and 10 kg of sick soil is used in per pot. T₂: Apply 10 mL of SQR-T037 fermentation liquor in each pot; T₃: Apply 20 g of microbial organic fertilizer containing SQR-T037 (simple mixture of 10 mL of SQR-T037 fermentation liquor and 20 g of organic fertilizer); T₄: Apply 20 g of microbial organic fertilizer generated from fermentation of SQR-T037. The materials are mixed well to ensure the concentration of the biocontrol strain in soil reaches 10⁵ cfu/g of dry soil. Five days after cucumber seedlings are transplanted, the morbidity is recorded. After 40 days, morbidity of CK₂ reaches 98.9%, whereas the incubation of SQR-T037 fermentation liquor, the microbial organic fertilizer containing SQR-T037 and the microbial organic fertilizer generated from fermentation of SQR-T037 all can effectively reduce the morbidity of wilt. T₄: The microbial organic fertilizer generated from fermentation of CK₂+SQR-T037 has the best control effect (Table 1, FIG. 2 A) and its morbidity is 7.3%.

### Wilt Control in the Soil for Continuous Cropping of Watermelon

The experimental soil is continuous cropping soil, with serious wilt. The treatment is as follows:
CK₁: Control; CK₂: Soil for continuous cropping of watermelon; T₁: CK₂+organic fertilizer; T₂: Microbial organic fertilizer produced through re-fermentation of CK₂+SQR-T037 and organic fertilizer;

Each treatment is repeated ten times. Each repetition uses 300 g of sick soil/cup. T₁: 3 g of ordinary organic fertilizer is applied in each cup; T₂: Apply 3 g of the organic fertilizer generated from re-fermentation of SQR-T037. The materials are mixed well. The concentration of SQR-T037 in soil is 10⁵ cfu/g of dry soil. Five days after watermelon seedlings are transplanted, the morbidity is recorded. After 21 days, the morbidity of CK₂ reaches 95.38%, whereas T₂: the microbial organic fertilizer generated from re-fermentation of CK₂+SQR-T037 can effectively reduce wilt morbidity and control it at 2.6%. (Table 2 and FIG. 2B)

The results indicate that the application of biocontrol strain SQR-T037 can not only control cucumber and watermelon wilt, greatly reduce the quantity of wilt pathogens (F. oxysporum) in soil, promote the changes of the microflora at rhizosphere of cucumber and watermelon, remarkably increase beneficial bacterial flora and remarkably reduce the total quantity of fungi and the quantity of pathogens (Table3), but also can remarkably increase the chlorophyl content, root system activity and solid content of cucumber and watermelon (Table 1 and Table 2).

After biocontrol strain is applied, the activity of the enzymes relating to systematic disease resistance of cucumber and watermelon is remarkably increased (Table 4).

### Field Experiment

SQR-T037 organic fertilizer was applied in the field that was infected by cucumber wilt in Yixing, Jiangsu in 2008.

Treatment of field experiment: 1. Apply organic fertilizer; 2. Apply the microbial organic fertilizer formed from the re-fermentation of SQR-T037 and organic fertilizer. The field with serious infection of wilt is randomly divided into 8 plots. In each treatment, four plots are selected randomly. Cucumber variety is “Jin Chun 4”. Fertilizer adopts hole application. The dosage of the bio-organic fertilizer is 30 kg/mu.

Data are collected from the field at regular intervals. Statistics of morbidity and microfloras is conducted. After final harvesting, the difference of biomass among different treatments is calculated. The application of the microbial organic fertilizer formed from the re-fermentation of SQR-T037 and organic fertilizer can remarkably reduce the occurrence of wilt, lower morbidity by 33.1%, and increase cucumber yield by 1.3 folds (Table 5).

Microbial organic fertilizer was applied in the field with serious watermelon wilt in Tongxu, Henan in 2007.

Treatment of field experiment: 1. Apply organic fertilizer; 2. Apply the microbial organic fertilizer formed from the re-fermentation of SQR-T037 and organic fertilizer. Select a field with serious infection of wilt, and randomly divide the field into 8 plots, and randomly select four plots for each treatment. The variety of watermelon is 8424. Fertilizer adopts hole application. The dosage of the bio-organic fertilizer is 30 kg/mu.

Data are collected from the field at regular intervals. Statistics on morbidity, disease index and microfloras is conducted. After final harvesting, the difference of biomass among different treatments is calculated. The application of the microbial organic fertilizer formed from the fermentation of SQR-T037 can remarkably reduce the occurrence of wilt. Comparing with the habitually applied fertilizers (non-grafted seedlings), morbidity is lowered by 20-40%, prevention and cure rate exceeds 95% and the yield of watermelon per mu increases by more than 600 kg (Table 5).

The present invention proceeds from microflora, and develops a microbial organic fertilizer product that can remarkably eliminate the occurrence of the wilt of continuous cropping cucumber and watermelon and achieves above 85% of controlling rate (field experiment). The mechanism of this fertilizer product is that the functional bacteria and their active carbon and nitrogen sources provided by this fertilizer product provide a very good condition for the cultivation of beneficial microflora in the soil with continuous cropping obstacle, and enable the soil to quickly restore and establish the ecology and food chain of exogenous functional bacterial organisms; on the other hand, this fertilizer product has high content of organic nitrogen and organic phosphor. These nutritive substances are beneficial to the growth of the crops in the soil with continuous cropping obstacle and greatly improve the seedling standing rate and tolerance of cucumber and watermelon.

TABLE 1
Chlorophyl Content Of Laminae, Activity Of Root System And
Dry Weight Of Plants Of The Potted Cucumber Under
Different Treatment

| | | Chlorophyl | Activity of | Dry weight |
|---|---|---|---|---|
| | Morbidity | content | root system | of plants |
| Treatment | (%) | (SPAD) | (mg TTC g⁻¹ h⁻¹) | (g) |
| CK1 | 0 | 35.27 ± 1.52 | 0.24 ± 0.04 | 26.87 ± 3.06 |
| CK2 | 98.9 | 27.93 ± 0.69 | 0.11 ± 0.02 | 12.77 ± 5.77 |
| T1 | 93.3 | 30.07 ± 1.17 | 0.14 ± 0.04 | 16.70 ± 3.06 |
| T2 | 25.6 | 35.27 ± 3.00 | 0.33 ± 0.04 | 30.86 ± 0.65 |
| T3 | 4.4 | 39.67 ± 0.94 | 0.40 ± 0.05 | 34.83 ± 2.57 |
| T4 | 0 | 46.80 ± 1.68 | 0.60 ± 0.03 | 36.61 ± 1.26 |

TABLE 2
Chlorophyl Content Of Laminae, Activity Of Root System And Dry
Weight Of Plants Of The Potted Watermelon Under Different Treatment

| | | Chlorophyl | Activity of | Dry weight |
|---|---|---|---|---|
| | Morbidity | content | root system | of plants |
| Treatment | (%) | (SPAD) | (mg TTC g⁻¹h⁻¹) | (g/cup) |
| CK1 | 0 | 63.38 ± 1.98 | 0.35 ± 0.06 | 1.32 ± 0.02 |
| CK2 | 95.38 | 56.01 ± 1.18 | 0.15 ± 0.03 | 0.68 ± 0.01 |
| T1 | 81.27. | 61.11 ± 5.26 | 0.21 ± 0.04 | 0.87 ± 0.03 |
| T2 | 2.60 | 92.28 ± 2.23 | 0.50 ± 0.05 | 3.03 ± 0.35 |

Note: The statistics of wilt morbidity follows international common rules: 0, the plant is healthy; 1, less than 10% of the plant wilts; 2, 11-20% of the plant wilts; 3, 21-50% of the plant wilts; 4, 50-100% of the plant wilts; 5, the plant dies. Calculation formula:
$Morbidity = \frac{\sum ( Level \times Plant quantity )}{Total plant quantity \times Top level} \times 100 \%$

TABLE 3
Difference Of Microbe Content On Root Surface Of The Potted
Watermelon Under Different Treatment

| | | | | Actino- | |
|---|---|---|---|---|---|
| Treatment | Pathogen | Trichoderma | Bacteria | myces | Fungi |
| | Quantity of the microbes at | | | | |
| | cucumber rhizosphere soil (cfu/g of soil) | | | | |
| CK₁ | 5.9 × 10² | 3.3 × 10³ | 6.2 × 10⁶ | 3.5 × 10⁴ | 8.1 × 10³ |
| CK₂ | 6.1 × 10⁴ | 8.8 × 10³ | 6.6 × 10⁶ | 5.2 × 10⁴ | 6.2 × 10⁴ |
| T1 | 7.9 × 10⁴ | 3.9 × 10³ | 7.7 × 10⁶ | 6.6 × 10⁴ | 8.9 × 10⁴ |
| T2 | 8.3 × 10³ | 5.5 × 10⁵ | 7.1 × 10⁶ | 5.8 × 10⁴ | 6.1 × 10⁵ |
| T3 | 3.2 × 10³ | 6.0 × 10⁵ | 8.3 × 10⁶ | 5.9 × 10⁴ | 6.9 × 10⁵ |
| T4 | 9.5 × 10² | 1.1 × 10⁶ | 8.9 × 10⁶ | 6.1 × 10⁴ | 1.9 × 10⁶ |
| | Quantity of the microbes at | | | | |
| | watermelon rhizosphere soil (cfu/g of soil) | | | | |
| CK₁ | 8.1 × 10² | 5.5 × 10³ | 5.1 × 10⁶ | 2.3 × 10⁴ | 3.7 × 10³ |
| CK₂ | 2.9 × 10⁴ | 5.9 × 10³ | 6.1 × 10⁶ | 5.1 × 10⁴ | 3.7 × 10⁴ |
| T1 | 3.1 × 10⁴ | 8.0 × 10³ | 7.9 × 10⁶ | 6.8 × 10⁴ | 6.1 × 10⁴ |
| T2 | 6.2 × 10³ | 5.7 × 10⁵ | 9.2 × 10⁶ | 9.9 × 10⁴ | 6.0 × 10⁵ |

TABLE 4
Changes Of Activity Of The Disease Resistance-Related
Enzymes At Root Under Different Treatment

| | PHENYL- | | |
|---|---|---|---|
| | ALANINE | | |
| | AMMONIA- | CHITINASE | |
| | LYASE | (μg of | β-1,3- |
| | (μg of | N-acetyl- | GLUCANASE |
| | cinnamic acid/ | glucosamine/ | (μg of glucose/ |
| | g of fresh | g of fresh | g of fresh |
| Treatment | weight/portion) | weight/portion) | weight/portion) |
| | Activity of disease resistance-related | | |
| | enzymes at cucumber root | | |
| CK1 | 16.20 ± 1.47 | 26.72 ± 1.68 | 106.39 ± 6.55 |
| CK2 | 36.00 ± 5.88 | 38.28 ± 1.25 | 153.26 ± 9.44 |
| T1 | 45.60 ± 5.88 | 39.89 ± 4.46 | 161.72 ± 17.91 |
| T2 | 78.60 ± 3.43 | 68.98 ± 5.32 | 332.29 ± 9.35 |
| T3 | 90.60 ± 5.39 | 87.18 ± 4.41 | 395.23 ± 35.02 |
| T4 | 112.20 ± 9.31 | 103.22 ± 11.12 | 615.08 ± 50.58 |
| | Activity of disease resistance-related | | |
| | enzymes at watermelon root | | |
| CK1 | 29.00 ± 6.21 | 29.78 ± 6.11 | 51.22 ± 3.32 |
| CK2 | 52.60 ± 5.35 | 39.89 ± 4.46 | 76.38 ± 8.82 |
| T1 | 58.60 ± 8.31 | 71.48 ± 8.33 | 81.28 ± 9.26 |
| T2 | 95.12 ± 8.82 | 112.25 ± 3.99 | 199.00 ± 12.10 |

TABLE 5
Effect Of Field Experiment

| | Effect of cucumber | | Effect of watermelon | |
|---|---|---|---|---|
| | field experiment | | field experiment | |
| | Yield | Morbidity | Yield | Morbidity |
| Treatment | (kg/mu) | (%) | (kg/mu) | (%) |
| Common composite | 960.92 | 35.1 | 3402 | 28.1 |
| fertilizer | | | | |
| Bio-organic fertilizer | 1229.50 | 2.0 | 4096 | 4.50 |

1. A biocontrol strain SQR-T037 against the wilt of continuous cropping cucumber and watermelon, which belongs to Trichoderma harzianum. It was collected by China General Microbiological Culture Collection Center (CGMCC) on Sep. 22, 2009, and the culture collection number is CGMCC No. 3308. The main biological characteristics are as follows: the optimum growth temperature on PDA culture medium is 30° C.; the diameter of colony can be reached 90 mm and it will cover the whole dish and generate yellow pigment in the culture medium when incubated for 96 hours; the conidiophore is in a shape of bottle; the conidium is oval, 2.3-3.5 μm long and 2.0-3.2 μm wide, and its surface is smooth; at the beginning its color is yellow green, then it changed into dark green slowly.

2. The microbial organic fertilizer is produced from the biocontrol strain according to claim 1.

3. The microbial organic fertilizer according to claim 2, wherein the content of biocontrol strain SQR-T037 is above 1×10⁸ cfu/g, the content of total nitrogen is 4˜5% (weight percent), above 90% of the total nitrogen is organic nitrogen, total nitrogen-phosphorus-kalium nutrient is 6˜10% (weight percent) and organic matter is 30˜35% (weight percent).

4. The microbial organic fertilizer according to claim 2, wherein it is produced by the following method:
(1) The strain SQR-T037 according to claim 1 is incubated to PDA culture to conduct liquid fermentation production under the following conditions: the initial pH range is 6.5-7.2; the culture temperature is 25-30° C.; DO: ventilation range is 30˜100% (v/v), 170 rpm; the fermentation time is 48 h; all mycelium pellets are broken into colony formation units of this strain in the late stage of fermentation, and the colony count of SQR-T037 in fermentation liquor is ≧1×109 cfu/mL;
(2) SQR-T037 fermentation liquor is respectively incubated to the mature livestock or poultry excrement compost and the mixture from microbial enzymatic hydrolysis of rapeseed meal at a dosage of 50 L/t. to conduct solid fermentation. The fermentation temperature is 30-50° C. During the fermentation, the material is turned over once a day. The fermentation is completed in 5-7 days. In the end, a solid preparation of biocontrol strain SQR-T037 is obtained. The colony count of this strain is above 1×10⁸ cfu/g;
(3) The solid microbial agent SQR-T037 made from livestock or poultry excrement compost (50-80% v/v) and the solid microbial agent SQR-T037 made from the mixture from enzymatic hydrolysis of rapeseed meal (20-50% v/v) are thoroughly mixed. The mixture is ripened 2-3 days. During the ripening, it is turned over twice. In the end, the microbial organic fertilizer is evaporated at temperature of below 50° C. till its water content is less than 30%. After packaging, the microbial organic fertilizer that prevents and eliminates the wilt of continuous cropping cucumber and watermelon is obtained.

5. The producing method of the microbial organic fertilizer according to claim 4, wherein the PDA culture used in step (1) is prepared by the following method (taking the preparation of 1 L of culture medium for example):
Unpeel 200 g of potato, cut it into small dices, and boil it in boiling water for 30 min, then filter the mixture, add 20 g of common sucrose into the filtrate, fix the volume at 1000 mL, adjust pH value to 6.5-7.0, and sterilize the liquor at 121° C. for 20 min.

6. The producing method of the microbial organic fertilizer according to claim 4, wherein the mature livestock or poultry excrement compost used in step (2) is characterized in that: the germination index of the mature livestock or poultry excrement compost is more than 98%, and the content of organic matter is ≧35%, as well as the content of organic nitrogen is 1.2-2% and water content is 25-30%.

7. The application of the microbial organic fertilizer according to claim 2.

8. The microbial organic fertilizer according to claim 3, wherein it is produced by the following method:
(1) The strain SQR-T037 according to claim 1 is incubated to PDA culture to conduct liquid fermentation production under the following conditions: the initial pH range is 6.5-7.2; the culture temperature is 25-30° C.; DO: ventilation range is 30˜100% (v/v), 170 rpm; the fermentation time is 48 h; all mycelium pellets are broken into colony formation units of this strain in the late stage of fermentation, and the colony count of SQR-T037 in fermentation liquor is ≧1×109 cfu/mL;
(2) SQR-T037 fermentation liquor is respectively incubated to the mature livestock or poultry excrement compost and the mixture from microbial enzymatic hydrolysis of rapeseed meal at a dosage of 50 L/t. to conduct solid fermentation. The fermentation temperature is 30-50° C. During the fermentation, the material is turned over once a day. The fermentation is completed in 5-7 days. In the end, a solid preparation of biocontrol strain SQR-T037 is obtained. The colony count of this strain is above 1×10⁸ cfu/g;
(3) The solid microbial agent SQR-T037 made from livestock or poultry excrement compost (50-80% v/v) and the solid microbial agent SQR-T037 made from the mixture from enzymatic hydrolysis of rapeseed meal (20-50% v/v) are thoroughly mixed. The mixture is ripened 2-3 days. During the ripening, it is turned over twice. In the end, the microbial organic fertilizer is evaporated at temperature of below 50° C. till its water content is less than 30%. After packaging, the microbial organic fertilizer that prevents and eliminates the wilt of continuous cropping cucumber and watermelon is obtained.

9. The producing method of the microbial organic fertilizer according to claim 8, wherein the PDA culture used in step (1) is prepared by the following method (taking the preparation of 1 L of culture medium for example):
Unpeel 200 g of potato, cut it into small dices, and boil it in boiling water for 30 min, then filter the mixture, add 20 g of common sucrose into the filtrate, fix the volume at 1000 mL, adjust pH value to 6.5-7.0, and sterilize the liquor at 121° C. for 20 min.

10. The producing method of the microbial organic fertilizer according to claim 5, wherein the mature livestock or poultry excrement compost used in step (2) is characterized in that: the germination index of the mature livestock or poultry excrement compost is more than 98%, and the content of organic matter is ≧35%, as well as the content of organic nitrogen is 1.2-2% and water content is 25-30%.

11. The application of the microbial organic fertilizer according to claim 3.
